(19) United States
(12) Patent Application Publication
(10) Pub. No.: US 2011/0275894 A1
(43) Pub. Date: Nov. 10, 2011

(54) CATHETER WITH CAMERA AND ILLUMINATOR AT DISTAL END

(76) Inventor: Robert A. Mackin, Flagstaff, AZ (US)

(21) Appl. No.: 13/107,119

(22) Filed: May 13, 2011

Related U.S. Application Data
(63) Continuation-in-part of application No. 11/865,256, filed on Oct. 1, 2007, now Pat. No. 7,942,813.
Application No. 11/865,256 is a division of application No. 10/775,904, filed on Feb. 10, 2004, now Pat. No. 7,297,105.

(51) Int. Cl. A61B 1/04

(52) U.S. Cl. 600/109

(57) ABSTRACT

A low cost camera and low cost light emitting diode are located at the distal end of a suction catheter to provide a real time image of the trachea or the gastrointestinal tract. Electrical conductors attached to or formed as part of the suction catheter terminate in an electrical connector exterior of the suction catheter for electrical connection with a power supply and a radio frequency or infrared transmitter for transmitting the signal generated by the camera. A radio frequency or infrared receiver receives the transmitted signal and provides it to a video monitor for displaying the image captured by the camera.

### CROSS-REFERENCE TO RELATED APPLICATIONS

This application is a continuation-in-part of a patent application entitled “Detachable Endotracheal Camera” filed Oct. 1, 2007 and assigned Ser. No. 11/865,256, now U.S. Pat. No. 7,942,813, which is a divisional of a patent application entitled “Endotracheal Camera”, filed Feb. 10, 2004 and assigned Ser. No. 10/775,904, now U.S. Pat. No. 7,297,105, and describing an invention by the present inventor.

### BACKGROUND OF THE INVENTION

1. Field of the Invention

The present invention relates to endotracheal tubes and, more particularly, to a catheter having an illuminator and a camera at its distal end coupled with electrical conductors and an electrical plug mating with a transmitter to transmit an image from the camera to a receiver for display on a video monitor, which catheter may be used for suction or for administering therapeutic fluid or liquid food.

2. Description of the Prior Art

The basic tenets attendant endotracheal tubes having an illuminator at the distal end are illustrated and described in U.S. Pat. No. 5,285,778 and relating to an invention by the present inventor; which patent is incorporated herein by reference. The endotracheal tube described therein includes an optical fiber extending through the endotracheal tube to a viewing lens at the distal end of the tube. An eye piece is attached to the proximal end of the optical fiber to permit viewing through the lens. Illumination of the area under inspection is provided by a high intensity light source extending via the endotracheal tube to an illumination port at the distal end.

### SUMMARY OF THE INVENTION

A catheter for insertion in the trachea or the gastrointestinal tract includes a light emitting diode at the distal end for providing illumination of the proximate tissue. A camera also disposed at the distal end captures an image of the illuminated tissue. Two sets of electrical conductors interconnect the diode and the camera with a plug(s) mating with a transmitter module that provides power to the diode and the camera and receives a signal from the camera reflective of the image captured. The transmitter module transmits the signal to a receiver for displaying the image on a video monitor for viewing. The catheter may be used for suction or for administering a fluid.

It is therefore a primary object of the present invention to provide a method for viewing tissue at the distal end of a catheter on a real time monitor with a wireless transmitter and receiver.

Another object of the present invention is to provide an inexpensive camera for recording an image at the distal end of a catheter.

A yet further object of the present invention is to provide a camera and a light emitting diode at the distal end of a catheter coupled with a low power transmitter to transmit an image recorded by the camera for viewing the image on a video monitor.

Still another object of the present invention is to provide a catheter coupled with a low power transmitter and receiver for transmitting an image at the distal end of the catheter to a video monitor for real time viewing.

A further object of the present invention is to provide a catheter with a light emitting diode and a small sized inexpensive camera at the distal end coupled through a detachably attached connector with a transmitter to transmit to a receiver an image captured by the camera for display on a video monitor.

A yet further object of the present invention is to provide a wireless transmission to a video monitor coupled with a light emitting diode and a camera recording an image at the distal end of a catheter using a low power radio frequency transmitter and receiver.

A still further object of the present invention is to provide a method for displaying an image real time on a video monitor by capturing the image to be displayed with a light emitting diode and a camera at the distal end of a catheter and transmitting the image by a radio frequency transmitter to a corresponding receiver to produce a signal for the video monitor.

A still further object of the present invention is to provide a method for viewing on a video monitor in real time an image at the distal end of a catheter using essentially an off the shelf low cost camera and a light emitting diode located at the distal end of the catheter and connected by electrical conductors with a wireless transmitter transmitting the captured image to a receiver connected to the video monitor.

These and other objects of the present invention will become apparent to those skilled in the art as the description thereof proceeds.

### BRIEF DESCRIPTION OF THE DRAWINGS

The present invention will be described with greater specificity and clarity with reference to the following drawings, in which:

FIG. 1 is a partial cross-sectional view of an endotracheal tube embodying fiber optics for transmitting an image;

FIG. 2 is a partial cross-section of the endotracheal tube;

FIG. 3 is a partial cross-sectional view illustrating placement within a patient of an endotracheal tube;

FIG. 4 illustrates a camera and a transmitter for attachment with a connector of an endotracheal tube;

FIG. 5 illustrates a receiver and an attached video monitor;

FIG. 6 illustrates a catheter that may be used as a suction catheter for clearing a passage of secretions;

FIG. 7 illustrates a representative cross-section of the catheter taken along lines 7-7, as shown in FIG. 6; and

FIG. 8 illustrates an end view of the catheter.

### DESCRIPTION OF THE PREFERRED EMBODIMENT

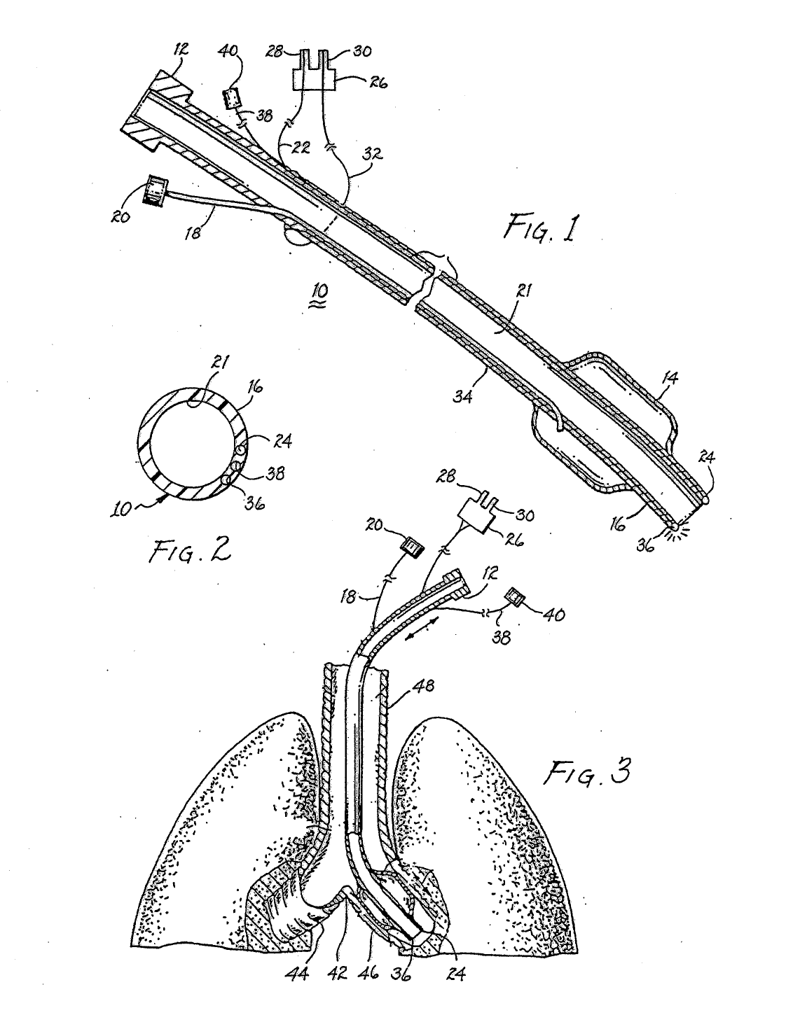

Referring to FIG. 1, there is shown an endotracheal tube 10 having a connector 12 for connection to a conventional ventilator to assist a patient's breathing function. The endotracheal tube includes an inflatable balloon 14 in proximity to its distal end 16. The inflatable balloon is inflated by a tube 18 connected through a connector 20 to a small syringe-like air pump after the endotracheal tube has been inserted into a patient's trachea.

Prior endotracheal tubes do not permit any visualization of a patient's tracheal and bronchial passages. If such visualization is needed, connector 12 is disconnected from the ventilator and a conventional bronchoscope is inserted down through hollow passage 21 of the endotracheal tube to allow a physician to determine if a lot of mucus is present in either lung or in either of the left or right stem main bronchi. If it is necessary to suction mucus out of either of the patient's lungs, a suctioning tube is inserted through hollow passage 21. The endotracheal tube may have to be disconnected from the ventilator to allow visualization in the trachea or the lungs or to allow suctioning of the mucus, blood, etc., if the endotracheal tube does not have a sealable side port through which the suctioning tube can be inserted.

When a skilled physician, often a pulmonologist, inserts an endotracheal tube into a patient, it would be desirable for a nurse to be able to easily monitor the position of the endotracheal tube in a patient's trachea to determine if its location has been shifted. If so, the nurse would know whether to call a physician to reposition the endotracheal tube. It would also be desirable to determine accurately the position of the endotracheal tube without requiring an x-ray of the patient.

Still referring to FIG. 1, endotracheal tube 10 includes an optical fiber, hereinafter referred to as fiber optic bundle 22, that extends through the endotracheal tube to a viewing lens 24 at distal end 16. The fiber optic bundle can be an inexpensive plastic optical fiber costing only a few dollars and embedded in the wall of the endotracheal tube. The fiber optic bundle is operatively connected to a connector 26 which includes two prongs 28, 30 of which prong 28 carries the fiber optic bundle. A second plastic optical fiber, hereinafter referred to as fiber optic bundle 32, extends through wall 34 of endotracheal tube 10 to an illumination port 36 at distal end 16.

FIG. 2 is a view of the distal end of endotracheal tube 10. A hollow tube 38 extends from a flushing inlet port connector 40 (see FIG. 1) and extends through the endotracheal tube so that a transparent saline flushing liquid can be forced through the tube to wash mucus away from viewing lens 24 and illumination port 36. Such mucus may collect thereon during insertion of the endotracheal tube into the patient's trachea or afterward.

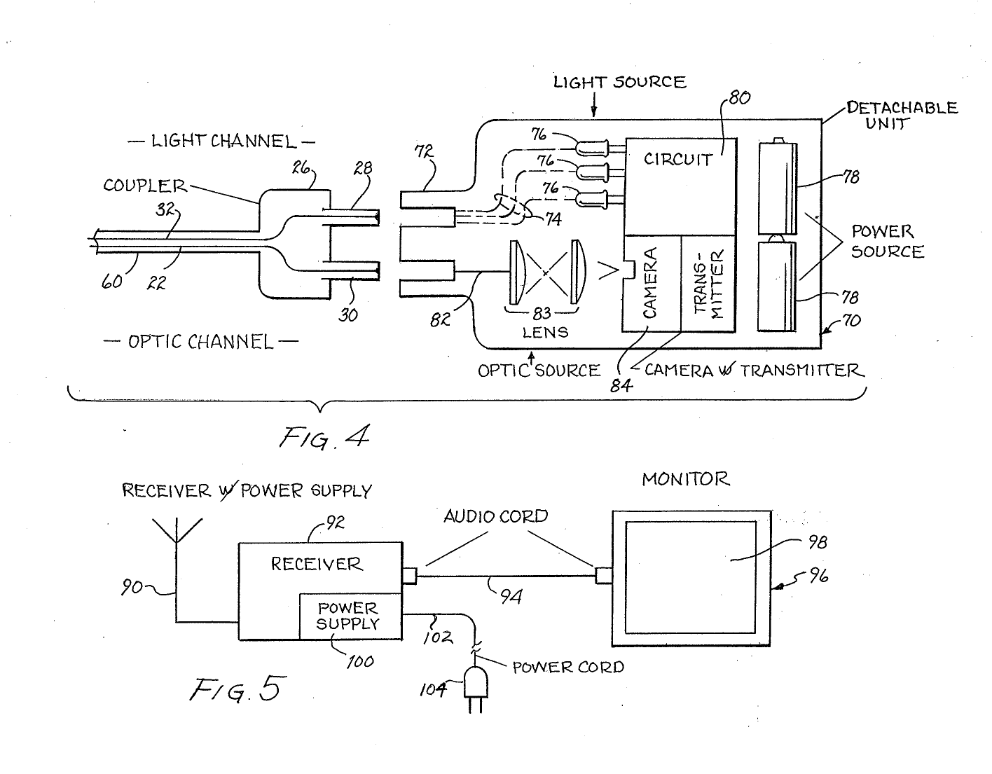

One major advantage of endotracheal tube 10 is that the carina (a cartilaginous structure) 42 (see FIG. 3) can be easily viewed during insertion of the endotracheal tube so that a nurse or a physician can readily determine how far into the patient's trachea to properly insert the endotracheal tube. This avoids the need for an x-ray process to determine if the endotracheal tube is properly inserted. As the endotracheal tube can become malpositioned in the patient and which would normally require a later x-ray to check for proper placement, direct visualization afforded by the present invention can avoid the need for such a repeat x-ray. Another advantage is that the nurse or physician can easily view the conditions in branches 44, 46 of trachea 48 to determine the presence of mucus or other condition and to determine whether there is a need for immediate suctioning of mucus, blood, etc., from either lung or the passages thereto.

Referring to FIG. 4, there is shown a male connector 26 having prongs 28, 30 extending therefrom. Fiber optic bundle 32 is in functional and operative engagement with prong 28 to transmit light from the end of the prong to illumination port 36 at distal end 16 of the endotracheal tube. Fiber optic bundle 22 is coupled with lens 24 at the distal end of the endotracheal tube to transmit light, that is an image, to the end of prong 30. As illustrated, fiber optic bundles 22 and 32 may be incased within a sheath 60.

A removable module 70 includes a female connector 72 for receiving prongs 28, 30 of connector 26. Upon mating of connectors 26, 72, fiber optic bundle 32 within prong 28 is placed in communication with fiber optic bundle 74, the latter being in communication with and receiving light from light emitting diodes 76. Electrical power for the light emitting diodes is provided by circuit 80 connected to batteries 78. Prong 30 of male connector 26 mates with female connector 72 to transmit light, that is, the image visible through lens 24 (see endotracheal tube 10) to convey the received light through a further fiber optic bundle 82 to a lens system 83. The lens system is interconnected with a small sized and relatively inexpensive electronic camera 84. Cameras suitable for this purpose cost less than $100.00 and can be found for less than $50.00 from commercial outlets. The camera is interconnected with a low power radio frequency transmitter 86 to transmit the images recorded by the camera. Transmitters of this type are readily available for less than $100.00 and may be found for less than $50.00 from commercial outlets.

As shown in FIG. 5, an antenna 90 is connected to a radio frequency receiver 92 and receives the images detected by camera 84 and transmitted by transmitter 86. The received image is conveyed via an electrical conductor or cord 94 to a video monitor 96. The video monitor includes a screen 98 for displaying the image recorded by camera 84. As illustrated, a power supply 100 provides power to receiver 92 and to video monitor 96 through an electrical conductor. Power to the power supply may be provided by an electrical conductor connected to a conventional plug 104 for engagement with a conventional wall socket.

In summary, the image conveyed from the lens at the distal end of the endotracheal tube is digitized and recorded by a camera. The image recorded by the camera is displayed real time on a video monitor through a wireless interconnection. The ease of a wireless transmission system in the confines of an operatory avoids the likelihood of a patient and attending health care providers from becoming entangled with cords and wires.

Moreover, presently used wires and cables extending to a video monitor creates a hazard of an attending health care provider inadvertently interfering with such wires and/or cables and causing repositioning or pulling out of the endotracheal tube. This hazard is completely avoided by the present invention due to the absence of such wires and/or cables.

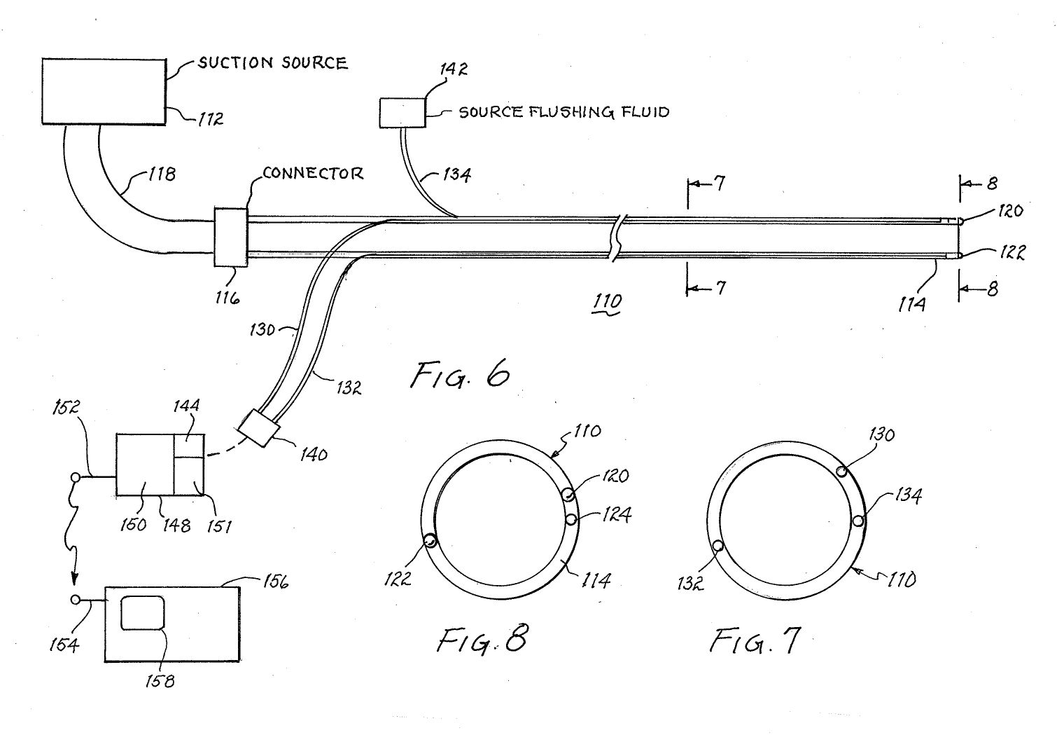

Referring to FIG. 6, there is shown a catheter 110 that may be used as a suction catheter or as a catheter for administering a therapeutic fluid or a liquid food. Hereinafter it will be described primarily as a suction catheter. The suction catheter may be flexible or rigid, depending upon the procedure to be performed. It includes a source of suction 112 (low pressure source) for drawing air, fluid and/or air and fluid mixture through the suction tube from distal end 114. The source of suction may also be referred to as a ventilator. The source of suction draws air, fluid or a mixture of air and fluid through the suction catheter to a depository (not shown). The suction catheter may be connected by a connector 116 to a conduit 118 extending from the source of suction.

Referring jointly to FIGS. 6, 7 and 8, details attendant distal end 114 and its function will be described. A low cost camera 120 is attached at the distal end of the suction catheter to capture and provide an image on command. To illuminate the area of interest of which an image is to be obtained, one or more light emitting diodes 122 is also attached at the distal end to provide illumination of the area/tissue of interest. To prevent contamination of the lens of camera 120 and remove obstructions from light emitting diode 122 that would impair radiation of light, a flush port 124 may be located to the distal end of the suction catheter. As illustrated in FIG. 8, camera 120, light emitting diode 122 and flush port 124 may be located within the side wall of the suction catheter. Alternatively, they may be attached to the interior wall or to the exterior wall of the suction catheter, depending upon manufacturing and cost considerations.

Electrical power for camera 120 along with electrical transmission of a signal reflective of the image captured by the camera are conveyed through electrical conductors 130, as particularly shown in FIG. 7. These conductors may be embedded in the side wall of suction catheter 110, as illustrated, or may be attached to either the interior or exterior wall of the suction catheter, depending upon manufacturing and cost considerations. Electrical energy for light emitting diode 122 is conveyed through electrical conductors 132. These electrical conductors may be embedded within the side wall of the suction catheter, as illustrated, or may be attached to the interior or exterior walls of the suction catheter. A conduit 134 conveys a fluid to flush port 124. This conduit may be embedded within the wall of the suction catheter or may be attached to the interior or exterior side walls of a suction catheter, depending upon manufacturing and cost considerations.

Referring to FIG. 6, electrical conductors 130 extend from suction catheter 110 and are terminated at a plug 140. Similarly, electrical conductors 132 are terminated at a plug, which plug may also be plug 140. Conduit 134 extends from the suction catheter and is in fluid communication with a source of flushing fluid 142.

A transmitter module 148 includes radio frequency transmitter 150, which could also be an infrared transmitter and an electrical connector 144 for electrically connecting with plug 140. The transmitter module provides electrical power, such as by batteries 151, to electrical conductors 130 and 132 to electrically energize camera 120 and light emitting diode 122. Additionally, it includes circuitry for receiving a signal from the camera reflective of the image captured by the camera. This signal is transmitted through antenna 152 to an antenna 154 in communication with a receiver 156.

This receiver may be adapted to respond to a radio frequency transmission or an infrared transmission from the transmitter. The transmission between antennas 152 and 154 includes the signal emitted by camera 120, which signal is manipulated to provide an image on video monitor 158. Accordingly, the image captured by the camera will be displayed on the video monitor on a real time basis.

With this invention, medical personnel can view in real time an image of the tissue attendant distal end 114 of suction catheter 110 during insertion, flushing and retraction, whether it be in the trachea or the gastrointestinal tract.

Although the use of a light emitting diode to provide illumination may be preferred, lighting the area of interest could also be provided by one or more fiber optic cables, as described above with respect to FIGS. 1, 2 and 4.

I claim:

1. Apparatus for displaying an image of tissue at the distal end of a suction catheter, said apparatus comprising:
(a) a source of light disposed at the distal end of said suction catheter for illuminating the tissue to be imaged, said source of light comprising at least one light emitting diode;
(b) a low cost camera disposed at the distal end of the suction catheter for generating an electrical signal representative of the image to be captured;
(c) a first set of electrical conductors extending from said camera for providing electrical power to said camera from a source of electrical power and for conveying the signal reflective of the image recorded by said camera;
(d) a second set of electrical conductors extending from said source of light for providing electrical power from a source of electrical power to said source of light;
(e) a low cost radio frequency transmitter for receiving the image from said camera through said first set of electrical conductors and for transmitting the signal reflective of the image recorded;
(f) a low cost radio frequency receiver for receiving the signal reflective of the image recorded;
(g) a video monitor coupled with said receiver for displaying the image reflective of the signal received by said receiver; and
(h) a source of flushing fluid connected through a conduit with a flushing port disposed at the distal end of said suction catheter.

2. Apparatus for displaying an image of tissue at the distal end of a suction catheter, said apparatus comprising:
(a) a source of light disposed at the distal end of said suction catheter for illuminating the tissue to be imaged and including a first set of electrical conductors for providing power to said source of light;
(b) a camera disposed at the distal end of said suction catheter for recording the illuminated image and including a second set of electrical conductors for providing power to said camera and for conveying a signal reflective of the image recorded;
(c) a transmitter for receiving the signal from said second set of electrical conductors and for transmitting the signal;
(d) a receiver for receiving the transmitted signal; and
(e) a display for displaying the image reflected by the signal received by said receiver.

3. The apparatus as set forth in claim 2 including a flush port disposed at the distal end of said suction catheter and a source of flushing fluid.

4. The apparatus as set forth in claim 2 wherein said source of light comprises at least one light emitting diode disposed at the distal end of said suction catheter.

5. The apparatus as set forth in claim 4 wherein the terminal ends of said first and second sets of electrical conductors are secured to a first plug and including a mating second plug coupled with said transmitter.

6. The apparatus as set forth in claim 5 including a source of flushing fluid and a flushing port disposed at the distal end of said suction catheter and a conduit interconnecting said source of flushing fluid with said flushing port for washing said camera and said source of light.

7. The apparatus as set forth in claim 6 wherein said first and second sets of electrical conductors and said conduit extend along said suction catheter.

8. The apparatus as set forth in claim 2 wherein said first and second sets of conductors are at least partially embedded in a side wall of said suction catheter.

9. The apparatus as set forth in claim 2 wherein said display is a video monitor.

10. The apparatus as set forth in claim 1 wherein said first and second sets of conductors are at least partially embedded in a side wall of said suction catheter.

11. The apparatus as set forth in claim 1 including mating plugs for interconnecting said first and second sets of electrical conductors with said transmitter.

12. The apparatus as set forth in claim 5 wherein said conduit is embedded in a side wall of said suction catheter.

13. The apparatus as set forth in claim 2 wherein said first and second sets of conductors are at least partially embedded in a side wall of said suction catheter.

14. The apparatus as set forth in claim 2 wherein said suction catheter is a flexible tube.

15. The apparatus as set forth in claim 2 wherein said suction catheter is a rigid tube.

16. The apparatus as set forth in claim 1 wherein a length of each of said first and second electrical conductors are embedded in the wall of said suction catheter.

17. A method for using a suction catheter, said method including the steps of:
(a) illuminating the distal end of the suction catheter with at least one light emitting diode;
(b) capturing an image of the tissue proximate the distal end of the suction catheter with a camera;
(c) providing electrical power to the at least one light emitting diode through a first set of electrical conductors;
(d) further providing electrical power to the camera and conveying a signal from the camera reflective of the image captured through a second set of electrical conductors;
(e) connecting the first and second sets of electrical conductors to a source of electrical power;
(f) conveying the signal from the second set of electrical conductors to a transmitter;
(g) transmitting the signal to a receiver;
(h) receiving the signal with the receiver; and
(i) displaying on a video monitor an image captured by the camera and represented by the signal received from the receiver.

18. The method as set forth in claim 17 wherein a length of each of the first and second electrical conductors are embedded in the wall of the suction catheter and including the step of coupling the terminal ends of the first and second electrical conductors with the transmitter.

19. The method as set forth in claim 17 wherein the transmitter is housed within a transmitter module and including the step of providing power to the at least one diode, the camera and the transmitter from batteries within the transmitter module.

20. Apparatus for displaying an image of tissue at the distal end of a catheter, said apparatus comprising:
(a) a source of light disposed at the distal end of said catheter for illuminating the tissue to be imaged and including a first set of electrical conductors for providing power to said source of light;
(b) a camera disposed at the distal end of said catheter for recording the illuminated image and including a second set of electrical conductors for providing power to said camera and for conveying a signal reflective of the image recorded;
(c) a transmitter for receiving the signal from said second set of electrical conductors and for transmitting the signal;
(d) a receiver for receiving the transmitted signal; and
(e) a display for displaying the image reflected by the signal received by said receiver.

21. The apparatus as set forth in claim 20 wherein said source of light comprises at least one light emitting diode disposed at the distal end of said catheter.

22. The apparatus as set forth in claim 21 wherein the terminal ends of said first and second sets of electrical conductors are secured to a first plug and including a mating second plug coupled with said transmitter.

23. The apparatus as set forth in claim 20 wherein said first and second sets of electrical conductors and said conduit extend along said catheter.

24. The apparatus as set forth in claim 20 wherein said first and second sets of conductors are at least partially embedded in a side wall of said catheter.

25. The apparatus as set forth in claim 20 wherein said display is a video monitor.

26. The apparatus as set forth in claim 20 including mating plugs for interconnecting said first and second sets of electrical conductors with said transmitter.

27. The apparatus as set forth in claim 20 wherein said catheter is a flexible tube.

28. The apparatus as set forth in claim 20 wherein said catheter is a rigid tube.
